(19) United States
(12) Patent Application Publication
(10) Pub. No.: US 2011/0245832 A1
(43) Pub. Date: Oct. 6, 2011

(54) WRAPPED CONNECTION BETWEEN CARBON FIBER SHAFT AND METAL PART

(75) Inventors: Helge Giersch, Kiel (DE); Ingo Stoltenberg, Probsteierhagen (DE)

(73) Assignee: STRYKER TRAUMA GMBH, Schonkirchen (DE)

(21) Appl. No.: 13/075,631

(22) Filed: Mar. 30, 2011

(30) Foreign Application Priority Data
Mar. 31, 2010 (EP) 10 158 572.7

(51) Int. Cl. A61B 17/16

(52) U.S. Cl. 606/80

(57) ABSTRACT

A reamer has a flexible carbon fiber composite shaft coupled to an interface element by wrapping a carbon fiber material so as to provide a reliable connection between the shaft and the interface element while maintaining flexibility of the shaft. The reaming device shaft has a mounting portion, the mounting portion having an end face and an outer surface. An interface element for mechanical coupling of an external device is provided and comprises a mounting portion, the mounting portion having an end face and an outer surface. The end face of the shaft mounting portion and the end face of the interface element mounting portion are facing towards each other and a carbon fiber wrapping is provided extending over both the outer surface of the shaft mounting portion and the outer surface of the interface element mounting portion

### CROSS REFERENCE TO RELATED APPLICATION

The present application claims priority from European Patent Application No. 10158572.7 filed Mar. 31, 2010, which is incorporated herein by reference

### BACKGROUND OF THE INVENTION

The present invention relates to a reaming device, and in particular to a reaming device providing a reliable connection between a carbon fiber composite shaft and an interface element.

Intramedullary nailing is the method of choice for the fixation of fractures in long bones, in particular bones in long extremities. To have full access to the intramedullary channel, a shaft of a reamer has to be flexible enough in a bending direction to bypass soft tissue and conform to bone curvature. The shaft also has to be rigid enough to convey torsion to the reamer head. Prior art reaming devices have a shaft design consisting of a helix in which residues can be trapped during the reaming procedure, so that the cleaning of the reaming device in hospitals prior to the next usage is complicated, in particular in a sterilization process. The adequate cleaning of the instrument in hospitals demands a great effort and takes a lot of time. Further, some hospitals are not prepared to clean such critical devices because of the great effort involved.

In some prior art reaming devices, a helix shaft is replaced by a shaft made of so called nitinol, which is a material having a high degree of elasticity (super elasticity) to provide enough flexibility. Nitinol is an acronym for NIckel TItanium Naval Ordnance Laboratory. Nitinol is the inter-metallic phase NiTi having a regular cubic crystal structure being different of the structure of titanium or nickel. Nitinol comprises about 55% nickel and about 45% titanium. Owing to the fact that the nitinol shaft is made of a single tube, the cleaning effort in the hospital is less exhausting. However, recent investigations have shown that the nitinol material has a catastrophic failure mode. In particular, some reports have pointed out that some breakages in multiple fragments of the nitinol shaft occurred during the reaming process during the intervention process in hospitals. Further, the nitinol material is a very expensive material.

EP 253526 relates to a shaft made of a composite material of filamentous fibers and an appropriate resin.

From US 2007/0015107, a root canal instrument having an abrasive coating and method for the production thereof is known, wherein the described root canal instrument has a core of a flexible elastic material having a shape memory, wherein the core furthermore has a coating with abrasive particles, wherein the core is made from a nickel-titanium alloy or from a plastic material, e.g. carbon fibre reinforced plastics material.

CH 668690 relates to a probe electrode cable for medical purposes, e.g. electro cardiogram test, using carbon fibre impregnated plastic insulating coating as a cover with a lead coupled to the test equipment.

US Patent Publication No. 2010/0239380 relates to a reaming device with a carbon fiber shaft , an interface element and a connecting agent. The disclosure of 2010/0239380 is incorporated herein by reference.

### BRIEF SUMMARY OF THE INVENTION

It may be seen as one aspect of the present invention to provide a more reliable reaming device.

This aspect of the present invention is solved by a reaming device comprising a shaft with a mounting portion, the mounting portion having an end face and an outer surface. An interface element is provided for mechanical coupling of an external device. The interface element comprises a mounting portion with the mounting portion having an end face and an outer surface. A carbon fiber wrapping is used over the end face of the shaft mounting portion and the end face of the interface element mounting portion which are facing towards each other. The carbon fiber wrapping extends over both, the outer surface of the shaft mounting portion and the outer surface of the interface element mounting portion. The shaft is made of a carbon fiber reinforced material. The end face of the shaft mounting portion and the end face of the interface element mounting portion may abut each other. The shaft mounting portion and the interface element mounting portion have corresponding outer diameters. The carbon fiber wrapping comprises a mold cover wherein the mold cover is a thermosetting mold cover. The interface element comprises a coupling portion for a reamer head as an external device. The coupling portion may comprise a dovetail. An outer surface of the coupling portion and the outer surface of the interface element mounting portion are coaxial, wherein the outer surface of the interface element mounting portion steps back over the outer surface of the coupling portion. The carbon fiber wrapping has an outer diameter corresponding to an outer diameter of the coupling portion. The outer surface of the interface element mounting portion comprises a recessed structure, wherein the recessed structure comprises a circumferential groove. The recess structure may comprise a knurled surface. The interface element may be made of a titanium alloy, wherein both the shaft and the interface element each have an elongated through bore, wherein both through bores align with each other.

According to an exemplary embodiment of the invention, a reaming device has a shaft with a mounting portion. The mounting portion has an end face and an outer surface. An interface element is provided for mechanically coupling an external device, such as, for example, a cutting tool. The interface element comprises a mounting portion, the mounting portion has an end face and an outer surface. A carbon fiber wrapping is utilized to connect the end face of the shaft mounting portion and the end face of the interface element mounting portion when facing towards each other. The carbon fiber wrapping extends over both the outer surface of the shaft mounting portion and the outer surface of the interface element mounting portion. Thus, a shaft of a reamer and an interface element may be reliably coupled and mounted by a carbon fiber wrapping, so that the connection between a shaft and an interface element can be manufactured without building up high internal material tensions, which may lead to an unpredictable material stress. Further, by providing a carbon fiber wrapping over both the outer surface of the shaft mounting portion and the outer surface of the interface element mounting portion, the total diameter of the reaming device may be kept low, so that the reaming device can be used in narrow spaces.

According to one exemplary embodiment of the invention, the shaft is made of a carbon fiber reinforced material. Thus, breakages can be avoided, as a carbon fiber reinforced material does not break into multiple fragments as is possible with a nitinol shaft. Further by using the same or similar material for the shaft and the wrapping, a reliable connection between the shaft and the wrapping may be established owing to the material compatibility.

According to one exemplary embodiment of the invention, the end face of the shaft mounting portion and the end face of the interface element mounting portion abut to each other. Thus, the transition portion between the shaft and the interface element can be kept short, so that the shaft device can be designed as a very compact device, in particular close to the interface element.

According to an exemplary embodiment of the invention, the shaft mounting portion and the interface element mounting portion have corresponding outer diameters. Thus, the carbon fiber wrapping may be wrapped around the outer surface of the shaft mounting portion and the outer surface of the interface element mounting portion without a step, so that sharp chamfers or grooves may be avoided, which chamfers or grooves may lead to a weakened connection owing to the general mechanical weakness of a chamfer.

According to an exemplary embodiment of the invention, the carbon fiber wrapping comprises a molded cover. Thus, the entire strength of the carbon fiber wrapping can be enforced. Further, the molded cover may seal the carbon fiber wrapping so as to avoid rough and porous surfaces generally leading to problems during the surgical intervention.

According to an exemplary embodiment of the invention, the molded cover is a thermosetting molded cover. Thus, the connection between the shaft and the interface element by a carbon fiber wrapping can be reinforced by the thermosetting process, generally allowing a reliable connection between a shaft and an interface element.

According to an exemplary embodiment of the invention, the interface element comprises a coupling portion for a reamer head as an external cutting device. Thus, a reamer head or a bore or drill head can be mounted to the interface element.

According to an exemplary embodiment of the invention, the coupling portion comprises a dovetail. Thus, a reliable connection between a reamer head and the interface element may be established. In particular when providing a dovetail transverse to the longitudinal extension of the interface element, pulling forces as well as pushing forces can be transmitted to or from the reamer head to the reaming device and vice versa. In particular, when providing the reamer head and the interface element with a through bore in a longitudinal direction, a securing or guiding wire can be inserted in order to avoid a lateral movement of the reamer head with respect to the interface element, while maintaining an easy assembling or disassembling when removing the guide wire.

According to an exemplary embodiment of the invention, an outer surface of a coupling portion and the outer surface of the interface element mounting portion are coaxial, wherein the outer surface of the interface element mounting portion steps back over the outer surface of the coupling portion. Thus, the carbon fiber wrapping can be placed in the stepped back outer surface of the interface element so as to not extend over the outer surface of the coupling portion. This allows for a smooth transition between the coupling portion and the wrapping.

According to an exemplary embodiment of the invention, the carbon fiber wrapping has an outer diameter corresponding to an outer diameter of the coupling portion. Thus, in particular when providing a step back geometry at the interface element, a smooth total surface can be provided starting from the outer diameter of the coupling portion via the outer diameter of the carbon fiber wrapping, and for example a smooth transition to the shaft. Thus, a smooth surface over the entire interface element and the transition portion to the shaft can be provided without any steps or recesses.

According to an exemplary embodiment of the invention, the outer surface of the interface element mounting portion comprises a recess structure. Such a recess structure provides a reliable mechanical coupling of the carbon fiber wrapping onto the interface element mounting portion, in particular, when using an impregnated carbon fiber wrapping. Thus, the force transition between the shaft and the interface element is not only based on a surface connection between the carbon fiber wrapping and the interface element, but also by a mechanical recess structure.

According to an exemplary embodiment of the invention, the recess structure comprises a circumferential groove. Thus, in particular axial forces can be transferred from the shaft to the interface element and vice versa, and a loosing of the interface element with respect to the shaft can be avoided, even if the surface connection between the wrapping and the interface element fails.

According to an exemplary embodiment of the invention, the recess structure comprises a knurled surface. The knurled surface provides a reliable mechanical coupling between the carbon fiber wrapping and the interface element, and at the same time increases the frictional forces between the shaft and interface element.

According to an exemplary embodiment of the invention, the interface element is made of a titanium alloy. Thus, in particular a lightweight and medical compatible material can be provided for the interface element.

According to an exemplary embodiment of the invention, the shaft and the interface element each have an elongated through bore, wherein both through bores aligning to each other. Thus, it is possible to insert a guide wire or securing wire into the aligning through bores. The guide wire may assist for a reliable targeting when reaming, wherein a securing wire may be used to secure a reaming head onto the coupling portion of the interface element.

It should be noted that the above features may also be combined. The combination of the above features may also lead to synergetic effects, even if not explicitly described in detail.

These and other aspects of the present invention will become apparent from and elucidated with reference to the embodiments described hereinafter.

### BRIEF DESCRIPTION OF THE DRAWINGS

FIG. 1 illustrates a shaft and an interface element as separate elements;

FIG. 2 illustrates a cross-sectional view of a shaft and an interface element being mounted together with a carbon fiber wrapping; and

FIG. 3 illustrates an end portion of a reaming device with a shaft and an interface element as well as a carbon fiber wrapping.

### DETAILED DESCRIPTION

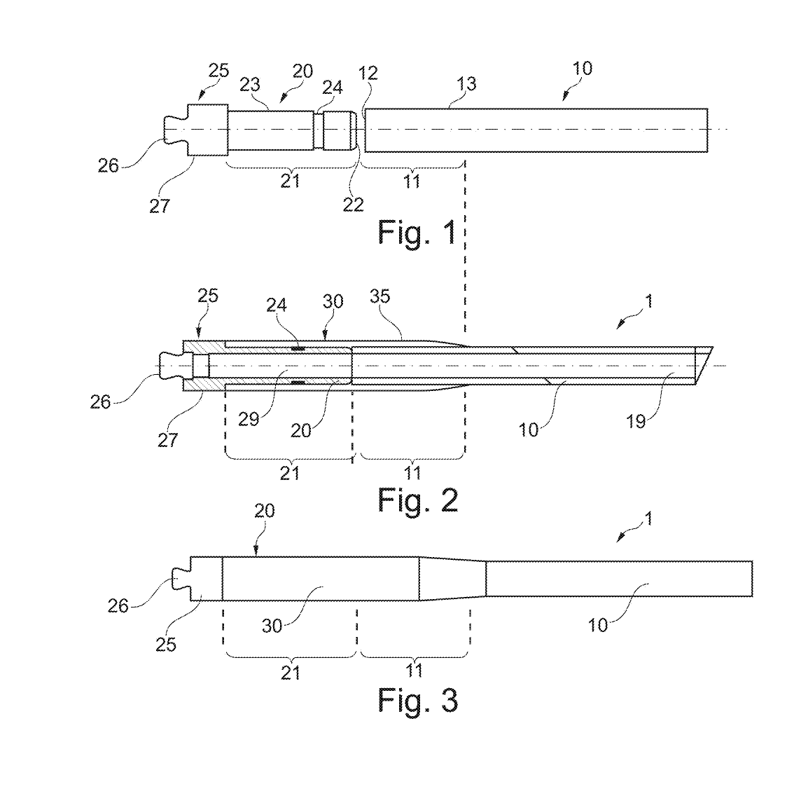

FIG. 1 illustrates a shaft 10 having an outer surface 13 and an end face 12. An end portion of the shaft 10 is used as a shaft mounting portion 11. This shaft mounting portion 11 serves for receiving a carbon fiber wrapping. The end face 12 is substantially perpendicular to the longitudinal direction of the shaft 10, however, it should be understood that the end face 12 may also be inclined or may have a surface structure like for example a waved or a toothed comb structure. The shaft 10 may be made of a carbon fiber material which is capable of carrying torque forces, and at the same time has a certain flexibility, which is required for surgical interventions.

An interface element 20 is also provided with an outer surface 23 and an end face 22. The end face 22 may be substantially perpendicular to the longitudinal extension of the interface element. However, end face 22 may also comprise a structure, for example corresponding to that of the end face 12 of the shaft 10 in order to have various kinds of interfering or mashing of end faces 12 and 22. Also the interface element 20 comprises an interface element mounting portion 21 for receiving a carbon fiber wrapping. The interface element 20 further comprises a coupling portion 25, which coupling portion 25 has an outer surface 27. An end portion of the coupling portion 25 may be provided with a coupling element 26, for example a dovetail. However, it should be noted that also any other coupling element 26 may be used. The interface element mounting portion 21 may comprise a surface structure 24, which may be for example a circumferentially extending groove and/or for example a knurled surface (not shown) for a reliable coupling of the carbon fiber wrapping, as will be described with respect to FIG. 2 in further detail.

FIG. 2 illustrates a cross-sectional view of an end portion of a reaming device, wherein end faces 12 of shaft 10 and 22 of interface element 20 abut each other. The carbon fiber wrapping 30 is wrapped around the outer surfaces 11, 21 of the shaft 10 and the interface element 20, respectively. Thus, a reliable connection between the shaft 10 and the interface element 20 can be established. The combination of a carbon fiber shaft 10 and for example a metal, in particular titanium alloy material for the interface element provide for a reliable combination of a flexible shaft and a form-stable and insensitive interface element for coupling a reamer head (not shown). The surface structure 24 of mounting portion 21 of interface element 20 provides for a reliable connection between the carbon fiber wrapping 30 and the surface of the interface element 20. In case, the shaft 10 is made of a carbon fiber composite material, the carbon fiber wrapping 30 and the carbon fiber material of the shaft 10 are compatible with each other, so that a reliable connection can be established, even if no surface structure is provided on the mounting portion 11 of the shaft 10. Nevertheless, also the mounting portion 11 of the shaft may be provided for with a surface structure.

As can be seen from FIG. 2, the carbon fiber wrapping 30 has an external dimension/diameter, so that the outer surface 11 of the coupling portion 25 smoothly aligns with the outer surface of a molded cover 35 of the carbon fiber wrapping 30. The wrapping can be in the form of, for example, PEEK impregnated carbon fiber wound around the outer surface of the coupling portion in a helical fashion. Thus, a smooth transition between the coupling portion 25 and the carbon fiber wrapping 30 can be established. The carbon fiber wrapping 30 can smoothly transit towards the shaft 10 so as to provide a smooth transition between the carbon fiber wrapping 30 and the surface of the shaft 10.

Both, the shaft 10 and the interface element 20 may be provided with a through bore 19, and 29, respectively. The aligning through bores 19 and 29 provide a possibility to put through a guide wire for a reliable targeting during intervention. Further, a securing wire can be put through the aligning through bores 19 and 29 as well a through bore of a reaming head (not shown) so as to avoid a lateral movement and losing of the reaming head being coupled by for example a dovetail.

FIG. 3 illustrates an outer view of the end portion of the reaming device 1, where it can be seen that the shaft 10, the interface element 20 together with its coupling portion 25, and the carbon fiber wrapping 30 together provide for a smooth surface without any steps or step transitions, so that a smooth total surface can be provided starting from the outer surface of a coupling portion via the outer surface of the carbon fiber wrapping towards an outer surface of the shaft 10.

### EXAMPLE

The process for making a carbon fiber composite (CFC) reamer shaft will now be described:

A prepreg fabric (Sigratex CE 8011-200-42-SGL Group) is cut into specific pieces for the shaft and the connection area by using a cutter; for example an Aristomat TL 1617. The pieces are then wound on a metal core by using an automatic rolling table. Cellophane tape is then wound over the CFC shaft to fit it and to withstand the expansion during heating. This is done by using a shrink film wrapper. The CFC shaft is then hardened in an oven and the cellophane tape is removed. The CFC shaft is then ground to a tolerance of ±0.05 mm and the core is removed. A small piece of CFC prepreg is wound on the machine connection side to later get a form fit for the injection molding part. A metal dovetail and the drill side of the CFC shaft is threaded over a second core and a CFC prepreg fabric is wound over both ends to fix the dovetail and CFC shaft together. Any cavities present are filled with epoxy. The CFC shaft with dovetail and machine side is then fixed in a mold made of two semicirclar parts. By closing the mold the form fit for the later injection moulding of the machine connection and a homogenous smooth transition between dovetail and CFC shaft will be pressed on the shaft. The mold is heated in an oven again to harden the expoxy. Then the machine connection is insert molded with a torque limiter made of SAN on the CFC shaft.

It should be noted that the term “comprising” does not exclude other elements and that the term “a” or “an” does not exclude a plurality. Also elements described in association with the different embodiments may be combined.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A reaming device comprising
a shaft with a mounting portion, the mounting portion having an end face and an outer surface;
an interface element for mechanical coupling of an external device, the interface element comprises a mounting portion, the mounting portion having an end face and an outer surface;
wherein the end face of the shaft mounting portion and the end face of the interface element mounting portion are facing towards each other; and
a carbon fiber wrapping extending over both the outer surface of the shaft mounting portion and the outer surface of the interface element mounting portion.

2. The reaming device according to claim 1, wherein the shaft is made of a carbon fiber reinforced material.

3. The reaming device according to claim 1, wherein the end face of the shaft mounting portion and the end face of the interface element mounting portion abut each other.

4. The reaming device according to claim 2, wherein the shaft mounting portion and the interface element mounting portion have corresponding outer diameters.

5. The reaming device according to claim 1, wherein the carbon fiber wrapping comprises a mold cover.

6. The reaming device according to claim 5, wherein the mold cover is a thermosetting mold cover.

7. The reaming device according to claim 1, wherein the interface element comprises a coupling portion for a reamer head as an external device.

8. The reaming device according to claim 7, wherein the coupling portion comprises a dovetail.

9. The reaming device according to claim 7, wherein an outer surface of the coupling portion and the outer surface of the interface element mounting portion are coaxial, wherein the outer surface of the interface element mounting portion steps back over the outer surface of the coupling portion.

10. The reaming device according to claim 7, wherein the carbon fiber wrapping has an outer diameter corresponding to an outer diameter of the coupling portion.

11. The reaming device according to claim 1, wherein the outer surface of the interface element mounting portion comprises a recessed structure.

12. The reaming device according to claim 11, wherein the recessed structure comprises a circumferential groove.

13. The reaming device according to claim 11, wherein the recess structure comprises a knurled surface.

14. The reaming device according to claim 1, wherein the interface element is made of a titanium alloy.

15. The reaming device according to claim 1, wherein both, the shaft and the interface element each have an elongated through bore, which through bores align with each other.
